(19) United States
(12) Patent Application Publication
(10) Pub. No.: US 2026/0177947 A1
(43) Pub. Date: Jun. 25, 2026

(54) MULTIFUNCTIONAL BELT

(71) Applicant: NITTA CORPORATION, Osaka-shi, Osaka (JP)

(72) Inventors: Takashi KANEDA, Yamatokoriyama-shi, Nara (JP); Shinji FURUKI, Yamatokoriyama-shi, Nara (JP); Koichi ARAI, Yamatokoriyama-shi, Nara (JP); Koichi TANABE, Yamatokoriyama-shi, Nara (JP)

(21) Appl. No.: 19/136,468

(22) PCT Filed: Dec. 8, 2023
(86) PCT No.: PCT/JP2023/044109

§ 371 Date: Jun. 6, 2025

(30) Foreign Application Priority Data
Dec. 9, 2022 (JP) 2022-197308

(51) Int. Cl. G03G 15/16; A61B 1/00; A61N 1/04; B66B 7/06; D07B 1/14; D07B 1/16; H01B 3/44

(52) U.S. Cl. CPC G03G 15/1615; A61B 1/00114; A61N 1/0492; B66B 7/062; D07B 1/147; D07B 1/162; H01B 3/441

(57) ABSTRACT

A multifunction belt including a flat belt-shaped belt main body made of a thermoplastic elastomer and a plurality of high strength core wires and a plurality of conductor core wires each embedded in the belt main body and extending in a longitudinal direction of the belt main body, in which in the plurality of high strength core wires, a part of the plurality of high strength core wires are embedded substantially at a center in a width direction of the belt main body, the plurality of conductor core wires are embedded offset from and outside the part of the plurality of high strength core wires in the width direction, and another part of the plurality of high strength core wires are embedded outside the plurality of conductor core wires in the width direction, and a tensile strength of the conductor core wires is smaller than a tensile strength of the high strength core wires.

### TECHNICAL FIELD

The present invention relates to a multifunction belt.

### BACKGROUND ART

In the related art, as a multifunction belt applied to an overhead transport vehicle, a slide fork, or the like provided in an automatic warehouse or the like, a multifunction belt including a plurality of high strength core wires and a plurality of conductor core wires embedded in a belt main body made of resin is known (for example, see Patent Literature 1). The multifunction belt disclosed in Patent Literature 1 has a configuration in which a plurality of high strength core wires such as aramid fibers are embedded substantially at a center in a width direction of a belt main body, and a plurality of conductor core wires such as aluminum wires, copper wires, and steel cords are embedded at end portions in the width direction of the belt main body.

The multifunction belt is connected to a control target such as an overhead transport vehicle or a slide fork via a take-up pulley, and performs movement of the control target, loading and unloading of a load, and the like by supplying power to the control target from the plurality of conductor core wires or transmitting an electrical control signal. Further, the multifunction belt is wound by the take-up pulley according to a movement position of the control target or the like. At this time, since a force acts on the multifunction belt in a tensile direction of the belt, the plurality of high strength core wires are embedded in order to have strength in the tensile direction.

### CITATION LIST

### Patent Literature

Patent Literature 1: JP2020-143772A

### SUMMARY OF INVENTION

### Technical Problem

However, the multifunction belt disclosed in Patent Literature 1 has a problem that when a force in the tensile direction acts on the multifunction belt, both ends of the multifunction belt are elongated to cause deformation such as waving, and the multifunction belt is likely to ride on a flange of the take-up pulley. In addition, since the force in the tensile direction strongly acts on the plurality of conductor core wires embedded in the end portions in the width direction of the belt main body, there is a possibility that the conductor core wires are deteriorated and disconnected due to metal fatigue or the like, and there is a problem that it is difficult to supply power to the control target and transmit a control signal.

An object of the present invention is to provide a multifunction belt in which deformation of an end portion does not occur and conductor core wires can be prevented from being damaged even when a force in a tensile direction is strongly applied.

### Solution to Problem

A multifunction belt according to the present invention includes: a flat belt-shaped belt main body made of a thermoplastic elastomer; and a plurality of high strength core wires and a plurality of conductor core wires each embedded in the belt main body and extending in a longitudinal direction of the belt main body, in which in the plurality of high strength core wires, a part of the plurality of high strength core wires are embedded substantially at a center in a width direction of the belt main body, the plurality of conductor core wires are embedded offset from and outside the part of the plurality of high strength core wires in the width direction, and another part of the plurality of high strength core wires are embedded outside the plurality of conductor core wires in the width direction, and a tensile strength of the conductor core wires is smaller than a tensile strength of the high strength core wires.

### Advantageous Effects of Invention

According to the present invention, since the high strength core wires are embedded outside the plurality of conductor core wires in the width direction, even when a force in a tensile direction acts on the multifunction belt, end portions of the multifunction belt are less likely to elongate, and deformation can be prevented. In addition, by making the tensile strength of the conductor core wires smaller than the tensile strength of the high strength core wires, even when a force in the tensile direction acts on the multifunction belt, the acting force can be borne more by the high strength core wires, thereby preventing damage such as disconnection from occurring in the conductor core wires.

### BRIEF DESCRIPTION OF DRAWINGS

FIG. 1 is a cross-sectional view illustrating a structure of a multifunction belt according to an embodiment of the present invention.

FIG. 2 is a graph illustrating elongation versus strength of high strength core wires and conductor core wires in the embodiment.

FIG. 3 is a cross-sectional view illustrating a structure of a multifunction belt according to a comparative example.

FIG. 4A is a photograph illustrating a multifunction belt according to the comparative example.

FIG. 4B is a photograph illustrating a multifunction belt according to an example.

FIG. 5 is a schematic view illustrating a method for measuring a pressure distribution acting on each of the multifunction belts in the comparative example and the example.

FIG. 6A is a plan view illustrating a structure of a flat pulley illustrated in FIG. 5.

FIG. 6B is a plan view illustrating a structure of a crown pulley illustrated in FIG. 5.

FIG. 7A is a photograph illustrating a state of an acting pressure distribution in the comparative example in which the flat pulley is used.

FIG. 7B is a photograph illustrating a state of an acting pressure distribution in the example in which the flat pulley is used.

FIG. 8A is a photograph illustrating a state of an acting pressure distribution in the comparative example in which the crown pulley is used.

FIG. 8B is a photograph illustrating a state of an acting pressure distribution in the example in which the crown pulley is used.

### DESCRIPTION OF EMBODIMENTS

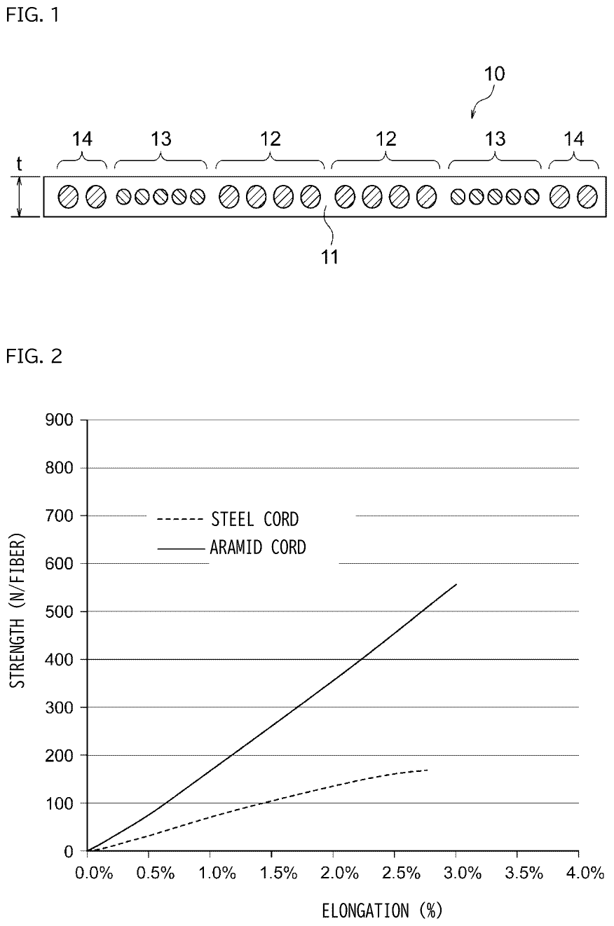

Hereinafter, embodiments of the present invention will be described. In the following description, members, structures, and the like that have already been described are denoted by the same reference numerals, and description thereof is omitted. FIG. 1 illustrates a cross-sectional view in a width direction of a multifunction belt 10 according to an embodiment of the present invention. The multifunction belt 10 is connected to a control target such as an overhead transport vehicle or a slide fork provided in an automatic warehouse such as a stacker crane, and performs movement of the control target, loading and unloading of a load in the automatic warehouse, and the like by supplying power to the control target or transmitting a control signal to the control target.

The multifunction belt 10 includes a belt main body 11, a plurality of high strength core wires 12 and 14, and a plurality of conductor core wires 13, and these core wires 12, 13, and 14 are embedded symmetrically in the width direction with respect to a center in the width direction of the belt main body 11. The belt main body 11 is a flat belt having a long flat belt-shaped belt main body 11 made of a thermoplastic elastomer. The thermoplastic elastomer constituting the belt main body 11 is preferably a urethane-based resin. A dynamic friction coefficient of a surface of the belt main body 11 with respect to a surface of a pulley formed of ultra-high molecular weight polyethylene is 0.1 or more and 0.2 or less. The plurality of high strength core wires 12 and 14 and conductor core wires 13 are embedded in the belt main body 11. In the present specification, the term “embedded” is not limited to a case in which the high strength core wires 12 and 14 and the conductor core wires 13 are completely embedded in the belt main body 11, and includes a case in which the high strength core wires 12 and 14 and the conductor core wires 13 are partially exposed from the belt main body 11. Specifically, a thickness t of the belt main body 11 is preferably 1.3 mm or more and 1.5 mm or less in consideration of winding by a pulley. A width of the belt main body 11 is preferably 20 mm or more and 40 mm or less in consideration of the number of the high strength core wires 12 and 14 and the conductor core wires 13.

In the plurality of high strength core wires 12 and 14, a part of the plurality of high strength core wires 12 are embedded substantially at a center in the width direction of the belt main body 11. In the present embodiment, eight high strength core wires 12 are embedded substantially at the center in the width direction of the belt main body 11. The number of the high strength core wires 12 embedded substantially at the center in the width direction of the belt main body 11 is not limited thereto, and may be less than eight or eight or more. As the plurality of high strength core wires 12 and 14, for example, high-strength and high-elasticity core wires such as aramid core wires, carbon core wires, polyparaphenylene benzobisoxazole (PBO) core wires, and high-strength glass core wires may be used. In the present embodiment, aramid core wires are employed.

The plurality of conductor core wires 13 are embedded from and outside the part of the plurality of high strength core wires 12 in the width direction of the belt main body 11. The plurality of conductor core wires 13 are embedded offset outward to both sides in the width direction from the plurality of high strength core wires 12 embedded substantially at the center. The plurality of conductor core wires 13 are, for example, steel core wires, soft copper core wires, copper alloy core wires, or aluminum core wires. In the present embodiment, steel core wires are employed. A tensile strength of the high strength core wires 12 and 14 at 0.1% elongation is preferably 15 N/wire or more, and a tensile strength of the conductor core wires 13 at 0.1% elongation is preferably 5 N/wire or less. A tensile elastic modulus of the high strength core wires 12 and 14 is preferably 3 times to 3.5 times a tensile elastic modulus of the conductor core wires 13. In the present embodiment, a wire diameter of the conductor core wires 13 is smaller than a wire diameter of the plurality of high strength core wires 12 and 14. Specifically, the wire diameter of the high strength core wires 12 and 14 is 0.4 mm or more and 1.2 mm or less, and the wire diameter of the conductor core wires 13 is 0.3 mm or more and 0.8 mm or less.

Another part of the plurality of high strength core wires 14 are embedded outside the plurality of conductor core wires 13. The plurality of high strength core wires 14 are made of the same material and have the same wire diameter as the plurality of high strength core wires 12 embedded substantially at the center of the belt main body 11, and are also subjected to surface treatment. In the present embodiment, four high strength core wires 14 are embedded outside the plurality of conductor core wires 13, but the number of embedded core wires is not limited thereto, and is set to an appropriate number according to a strength required for end portions of the multifunction belt 10.

The multifunction belt 10 is made of, for example, a urethane-based resin composition, and is manufactured by continuously melt-extruding the composition into a sheet shape from a tip of die (T-die) of an extrusion molding machine, and pouring the molten urethane-based resin into a cavity formed between a mold roll and a steel band. At this time, the conductor core wires 13 and the high strength core wires 12 and 14 are drawn together with the pouring, and the urethane-based resin layer in which the core wires are embedded is molded, whereby the multifunction belt 10 can be manufactured. In the multifunction belt 10, the high strength core wires 12 and 14, the conductor core wires 13, and the belt main body 11 are integrated without using an adhesive.

According to the present embodiment, the following effects are obtained. Since the high strength core wires 14 are embedded outside the plurality of conductor core wires 13 in the width direction, even when a force in a tensile direction acts on the multifunction belt 10, the end portions of the multifunction belt 10 are less likely to extend, and deformation can be prevented. In addition, by making the tensile strength of the conductor core wires 13 smaller than the tensile strength of the high strength core wires 12 and 14, even when a force in the tensile direction acts on the multifunction belt 10, the acting force can be borne more by the high strength core wires 12 and 14, thereby preventing damage such as disconnection from occurring in the conductor core wires 13.

By making the wire diameter of the conductor core wires 13 smaller than the wire diameter of the high strength core wires 12 and 14, when the multifunction belt 10 is wound around a take-up pulley and wound up thereto, or the like, a pressure in an out-of-plane direction acting on the multifunction belt 10 can be reduced from acting on the conductor core wires 13, and the conductor core wires 13 can be prevented from being damaged by disconnection or the like due to metal fatigue or the like.

### EXAMPLE

Next, examples of the present invention will be described. The present invention is not limited to these examples and includes various modifications.

### (1) Experimental Example 1

The strength of the high strength core wires 12 and the conductor core wires 13 used in the multifunction belt 10 according to a first embodiment was measured according to JIS L1013 chemical fiber filament yarn test method (2010). A load-elongation curve was obtained with a tensile tester (manufactured by Shimadzu Corporation) at a grip interval of 150 mm and a tensile speed of 300 mm/min. A load value at break was taken as a strength (N/fiber), and an elongation at break divided by an initial sample length was taken as an elongation rate (%). A relation between the strength (N/fiber) and the elongation (%) of the high strength core wires 12 and the conductor core wires 13 in the multifunction belt 10 was compared. An aramid cord (wire diameter: 0.7 mm) was used for the high strength core wires 12, and a steel cord having a wire diameter of 0.36 mm was used for the conductor core wires 13.

Experimental results are illustrated in FIG. 2. As can be seen from FIG. 2, it was confirmed that the aramid cord is a material having a small elongation with respect to the strength and being difficult to elongate with respect to a tensile force. Meanwhile, it was confirmed that the steel cord is a material having a large elongation with respect to the strength and prone to metal fatigue and other problems when a tensile force is applied. Therefore, it was confirmed that, by sandwiching the plurality of conductor core wires 13 between the plurality of high strength core wires 12 and 14 as in the embodiment, even when a force in the tensile direction acts on the multifunction belt 10, the plurality of conductor core wires 13 are less likely to elongate, and thus metal fatigue can be prevented and occurrence of disconnection or the like can be prevented.

### (2) Experimental Example 2

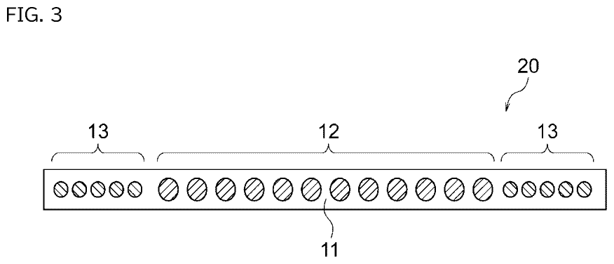

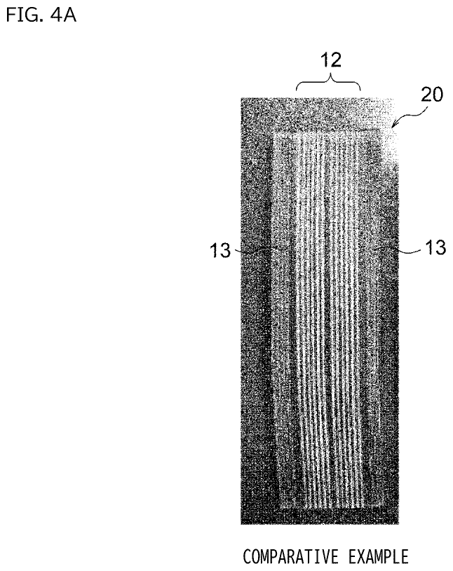

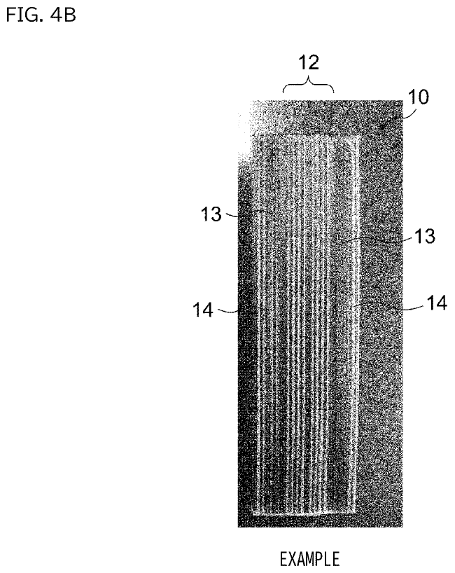

A pressure distribution generated in the multifunction belt due to the tensile force acting on the multifunction belt when the multifunction belt was attached to a take-up pulley and wound onto the take-up pulley was measured. Specifically, a pressure distribution generated in a multifunction belt 20 illustrated in FIG. 3 as a comparative example was measured in comparison with the multifunction belt 10 described in the embodiment. In the multifunction belt 20 according to the comparative example, twelve high strength core wires 12 were embedded in the central portion, and a plurality of conductor core wires 13 were embedded in both side end portions of the high strength core wires 12. FIG. 4A illustrates a photograph of the multifunction belt 20 as a comparative example as viewed from above, and FIG. 4B illustrates a photograph of the multifunction belt 10 as an example as viewed from above.

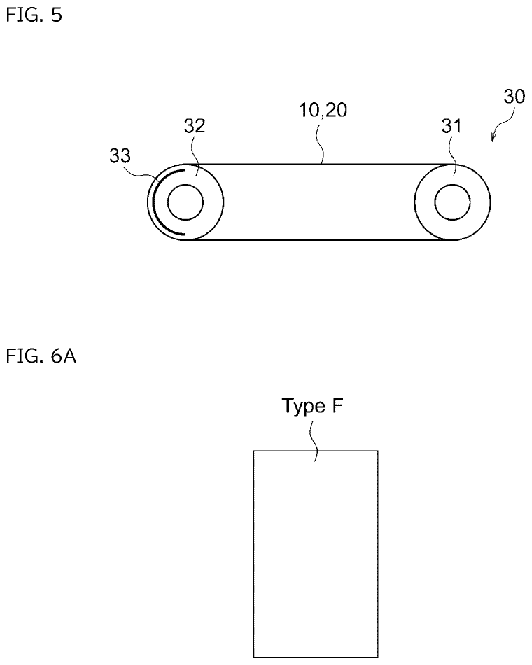

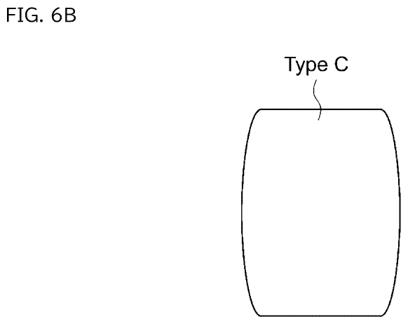

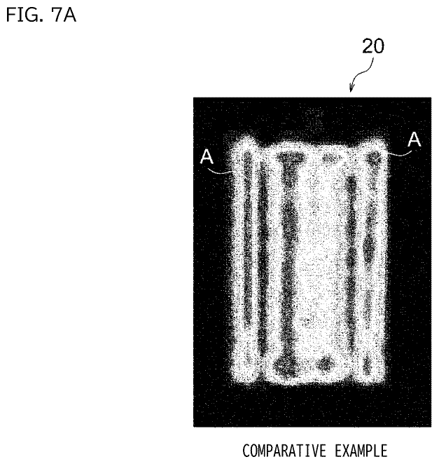

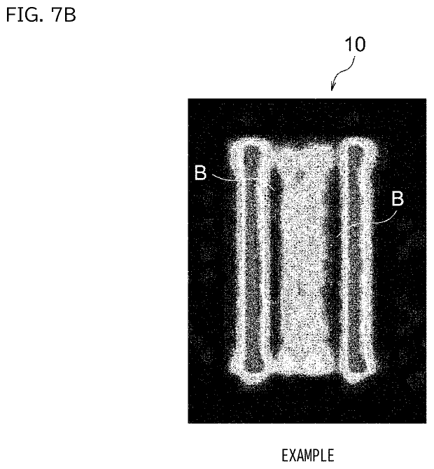

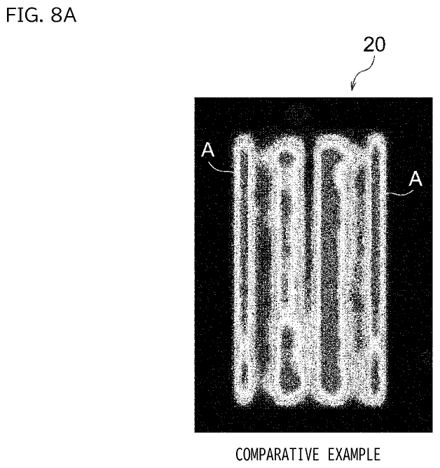

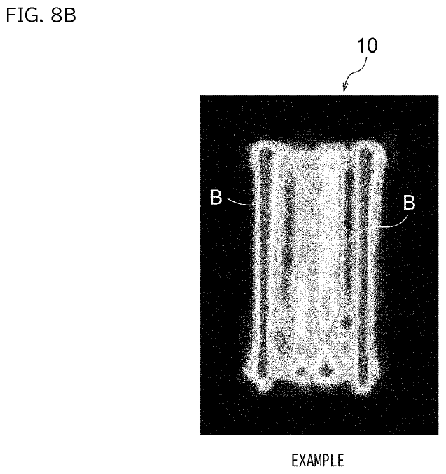

Regarding the measurement method, as illustrated in FIG. 5, the multifunction belts 10 and 20 were wound with a tension of 300 N around a wide-width running tester 30 including a drive wheel 31, a driven wheel 32, and a pressure distribution sensor 33 (Tactile Sensor iscan100 manufactured by Nitta Corporation), which serves as a take-up pulley, and the pressure distribution detected by the pressure distribution sensor 33 was measured while driving the drive wheel 31. The diameter of each of the drive wheel 31 and the driven wheel 32 was 32 mmφ, and the width of each of the multifunction belts 10 and 20 was 30 mm. Further, as the drive wheel 31 and the driven wheel 32, a flat pulley Type F illustrated in FIG. 6A and a crown pulley Type C illustrated in FIG. 6B were used for measurement. The radius of curvature of the crown of the crown pulley Type C was 770 mmR, and the height of the crown was 0.3 mm. Results are illustrated in FIGS. 7A and 7B and FIGS. 8A and 8B.

When the flat pulley Type F was used as the drive wheel 31 and the driven wheel 32, measurement results as illustrated in FIGS. 7A and 7B were obtained. As illustrated in FIG. 7A, in the multifunction belt 20 according to the comparative example, it was confirmed that the pressure distribution measured in a region A in which the plurality of conductor core wires 13 are embedded is high, and the load acting on the plurality of conductor core wires 13 is high. Meanwhile, as illustrated in FIG. 7B, in the multifunction belt 10 according to the example, since the plurality of high strength core wires 14 are embedded outside the plurality of conductor core wires 13 in the width direction, it was confirmed that the pressure distribution measured in a region B in which the plurality of conductor core wires 13 are embedded is low, and the load acting on the plurality of conductor core wires 13 is reduced.

Even when the crown pulley Type C is used as the drive wheel 31 and the driven wheel 32, as illustrated in FIGS. 8A and 8B, it was confirmed that the pressure distribution in the region A in which the plurality of conductor core wires 13 are embedded is high in the multifunction belt 20 according to the comparative example, and the pressure distribution in the region B in which the plurality of conductor core wires 13 are embedded is low in the multifunction belt 10 according to the example. As described above, in the multifunction belt 10 according to the example, since the high strength core wires 14 are embedded in the end portions in the width direction of the belt main body 11, even when a tensile force acts on the multifunction belt 10, a high pressure distribution is not generated in the plurality of conductor core wires 13. Therefore, it was confirmed that the load acting on the plurality of conductor core wires 13 can be reduced and damage such as disconnection can be prevented from occurring in the plurality of conductor core wires 13.

### REFERENCE SIGN LIST

10 multifunction belt
11 belt main body
12, 14 high strength core wires
13 conductor core wires

1. A multifunction belt comprising:
a flat belt-shaped belt main body made of a thermoplastic elastomer; and
a plurality of high strength core wires and a plurality of conductor core wires each embedded in the belt main body and extending in a longitudinal direction of the belt main body, wherein
in the plurality of high strength core wires, a part of the plurality of high strength core wires are embedded substantially at a center in a width direction of the belt main body, the plurality of conductor core wires are embedded offset from and outside the part of the plurality of high strength core wires in the width direction, and another part of the plurality of high strength core wires are embedded outside the plurality of conductor core wires in the width direction, and
a tensile strength of the conductor core wires is smaller than a tensile strength of the high strength core wires.

2. The multifunction belt according to claim 1, wherein
the plurality of conductor core wires include a control signal line configured to electrically control a control target serving as a connection destination.
